(19) United States
(12) Patent Application Publication
(10) Pub. No.: US 2020/0000917 A1
(43) Pub. Date: Jan. 2, 2020

(54) METHODS OF PHOTOCHEMICAL TREATMENT FOR WOUND HEALING

(71) Applicant: The General Hospital Corporation, Boston, MA (US)

(72) Inventors: Michael McCormack, Medford, MA (US); Robert Redmond, Newton Centre, MA (US); William Gerald Austen, Jr., Weston, MA (US)

(21) Appl. No.: 16/487,517

(22) PCT Filed: Feb. 22, 2018
(86) PCT No.: PCT/US18/19286

§ 371 Date: Aug. 21, 2019

Related U.S. Application Data
(60) Provisional application No. 62/484,594, filed on Apr. 12, 2017.
(60) Provisional application No. 62/462,013, filed on Feb. 22, 2017.

(51) Int. Cl. A61K 41/00; A61K 35/12; A61K 38/18

(52) U.S. Cl. CPC A61K 41/0057; A61K 38/18; A61K 35/12

(57) ABSTRACT

A method for improving healing of a wound includes delivering an activating agent to the wound and irradiating the wound with an electromagnetic radiation source. The method also includes activating the activating agent, in response to the irradiation, to cause extracellular matrix crosslinking throughout the wound.

### CROSS-REFERENCE TO RELATED APPLICATIONS

This application is based on, claims priority to, and incorporates herein by reference in their entirety, U.S. Provisional Patent Application Ser. No. 62/462,013 filed on Feb. 22, 2017 and U.S. Provisional Patent Application Ser. No. 62/484,594 filed on Apr. 12, 2017.

### BACKGROUND

Wound healing is a dynamic process that includes four overlapping phases: hemostasis, inflammation, proliferation, and remodeling. During hemostasis, constriction of the damaged vessels and clot formation physically limit blood loss. In the inflammatory phase, leukocytes and then monocytes accumulate to combat infection in the wounded tissue. In this phase, multiple cytokines and growth factors are released to the wound area and contribute to fibroblast migration, differentiation and activity. During the proliferative phase, fibroblasts deposit new extracellular matrix and collagen and differentiate into myofibroblasts, which facilitate healing by reducing the size of the wound (e.g., contracture). In this phase, cells generally undergo apoptosis as their roles near completion. In the final remodeling phase, re-organization of the closed wound environment occurs until repair is completed, where unneeded cells are removed by apoptosis.

Optimal wound healing includes complete restoration of tissue function and structure. However, many wounds are characterized by incomplete restoration of structure and function. For example, scarring may result when the healing process does not stop as it should, such as when tissue fails to reach a normal cell density and there is an improper balance between collagen deposition and degradation (e.g., cells do not undergo apoptosis when they should). Furthermore, when contraction continues for too long, it can lead to permanent disfigurement and loss of function.

Natural healing of large surface area wounds, for example, from burns, trauma, or iatrogenic injury, often results in significant contracture and scarring. More specifically, large surface area wounds that undergo secondary intention healing (that is, healing where wound edges are not brought together) take longer to heal than wounds undergoing primary intention healing (that is, closed wounds, such as closed surgical incisions). Given the significant amount of rebuilding required in secondary intention healing, the collagen structure in the wound is often disorganized, resulting in thin collagen fibers that are haphazardly organized. Scarring also results from overactive fibroblasts, and too many myofibroblasts that are active for too long results in significant contracture. As a result, the healed wound often does not match the normal tissue coloring, structure, and/or function of surrounding tissue.

Therefore, it would be desirable to provide systems and methods for improving wound healing of large surface area wounds with less contracture.

### SUMMARY

The systems and methods of the present disclosure overcome the above and other drawbacks by providing a method and system for improving healing of an epithelial tissue wound. The method includes delivering an activating agent to the wound and irradiating the wound with an electromagnetic radiation source. The method also includes activating the activating agent, in response to the irradiation, to cause crosslinking of the extracellular matrix in the wound.

The foregoing and other advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a flow diagram illustrating a method in accordance with aspects of the present disclosure.

FIG. 2 is a schematic view of a system in accordance with aspects of the present disclosure.

FIG. 3 is a diagram illustrating treatment steps in accordance with methods of the present disclosure.

FIG. 4 is a chart illustrating percentage of skin area around a wound perimeter as a function of days after wound creation in a control group and in a test group treated in accordance with methods of the present disclosure.

FIGS. 5A and 5B illustrate tissue harvested from a control group and a test group treated in accordance with methods of the present disclosure, respectively, at 7 days, 21 days, and 42 days after wound creation.

### DETAILED DESCRIPTION

The disclosure provides a system and method for improving wound healing through photochemical crosslinking of tissue collagen and other structural proteins. This photochemical treatment system and method can be used to manipulate the wound healing response in order to reduce scarring and contracture typically associated with large surface area wounds. For example, the system includes a mechanism to deliver an activating agent to the target wound, and an energy source to irradiate the target wound with electromagnetic radiation. The energy source may include an electromagnetic radiation source that activates the activating agent, which manipulates the wound healing process through extracellular matrix crosslinking, blunting the fibrotic response, and thereby decreasing contracture and associated morbidities. The system and method described herein may be applicable to wounds of the epithelium such as full thickness skin wounds or partial thickness skin wounds, any raw, wounded, or injured skin tissue, or open wounds containing a tissue graft. The system and method described herein may further be applicable to wounds of any epithelial tissue, such as wounds caused by resections or dissections of such tissue, including, but not limited to, endoscopic submucosal dissection (ESD) and endoscopic mucosal resection (EMR).

FIG. 1 illustrates a method 10 according to one aspect of the disclosure. Generally, the method 10 may include photochemical treatment of a wound. In one non-limiting example, such photochemical treatment may be photochemical tissue passivation. More specifically, the method 10 includes delivering an activating agent to the wound at process block 12. Once the agent is delivered to the wound, the target tissue is irradiated at process block 14. As one non-limiting example, the irradiation at process block 14 may be performed using an electromagnetic radiation source. In particular, as will be described, the irradiation at process block 14 is specifically performed to activate the activating agent delivered at process block 12 to cause, at process block 16, matrix crosslinking within the target wound. The crosslinking improves the natural wound healing response at process block 18, resulting in healed tissue that better matches structure and function of surrounding tissue.

With respect to process block 12, an activating agent is delivered to a wound. Generally, an activating agent is a chemical compound that produces a chemical effect upon photoactivation or a chemical precursor of a compound that produces a chemical effect upon activation. For example, the activating agent may be a photochemical agent, such as a photosensitizer or photoactive dye. In one specific application, the activating agent can be Rose Bengal. In a further application, the activating agent can be 0.1% Rose Bengal in a saline solution. In other applications, the activating agent may be selected from the group consisting of xanthenes, flavins, thiazines, porphyrins, expanded porphyrins, chlorophylls, phenothiazines, cyanines, mono azo dyes, azine mono azo dyes, rhodamine dyes, benzophenoxazine dyes, oxazines, and anthroquinone dyes. In yet other applications, the activating agent may selected from the group consisting of Rose Bengal, erythrosine, riboflavin, methylene blue (“MB”), Toluidine Blue, Methyl Red, Janus Green B, Rhodamine B base, Nile Blue A, Nile Red, Celestine Blue, Remazol Brilliant Blue R, riboflavin-5-phosphate (“R-5-P”), N-hydroxypyridine-2-(I H)-thione (“N-HTP”) and photoactive derivatives thereof. Furthermore, in some applications, the activating agent may be a chemical crosslinking compound.

Delivery at process block 12 may include, but is not limited to, staining, painting, brushing, spraying, dripping, injecting, or otherwise applying the activating agent to a surface of the wound. According to one example, the activating agent may be applied to a surface of the wound using one or more applicators, such as sponges, brushes, and cotton tip applicators. The amount of activating agent applied to the wound using such applicators can depend on the type of wound and, more specifically, the amount of collagen and other structural proteins in the wound. According to another example, an applicator can be a material, such as a pre-treated bandage, containing the activating agent, so that the applicator can be placed on the wound surface to transfer the activating agent to the wound. Additionally, in some aspects, the delivery mechanism may further include a tool for delivering the applicator to the wound, such as an endoscope, guide needle, or other instrument.

Referring now to process block 14, the wound, containing the activating agent may be irradiated, for example using an energy source. In some aspects, the energy source may be an electromagnetic radiation source configured to emit light at an appropriate energy and wavelength, and for a suitable duration, to cause agent activation. For example, the electromagnetic radiation source can be configured to irradiate the wound at an irradiance of less than about one watt per centimeter squared (W/cm²). In other applications, however, light can be delivered at an irradiance between about 0.5 W/cm² to about five W/cm², preferably between about one W/cm² and about three W/cm², and more preferably between about 0.5 W/cm² and about one W/cm². With respect to energy, in one aspect, the electromagnetic radiation source may be configured to emit radiation at 60 Joules per centimeter squared. In some aspects, a fluence range may between about 30 and about 120 Joules per centimeter squared. Also, the electromagnetic radiation source can emit light at the wound for an appropriate duration based on the activating agent and wound type. Generally, the duration of irradiation can be brief and sufficient to allow crosslinking within the tissue. In some applications, the wound is irradiated for a duration of about one minute to about thirty minutes. In other applications, the wound is irradiated for a duration of less than about five minutes.

Generally, the electromagnetic radiation source can be configured to emit energy, e.g., light, having a wavelength in the visible range or portion of the electromagnetic spectrum. In some aspects, the electromagnetic radiation source can be a low-energy visible-light emitter, for example, configured to emit monochromatic or polychromatic light. In other aspects, however, the electromagnetic radiation source may emit radiation other than visible light, such as radiation in the ultraviolet or infrared regions of the electromagnetic spectrum. Suitable electromagnetic radiation source examples include, but are not limited to, commercially available lasers, optical fibers, waveguides, lamps, one or more light-emitting diodes (“LEDs”), or other sources of electromagnetic radiation. In one specific example, the electromagnetic radiation source can be an LED array. In another example, the electromagnetic radiation source can be a KTP (potassium titanyl phosphate) laser.

Furthermore, the electromagnetic radiation source can emit radiation at an appropriate wavelength that activates the type of activating agent used. More specifically, the wavelength of light can be chosen so that it corresponds to or encompasses the absorption spectrum of the activating agent. For example, when Rose Bengal is the activating agent used, the electromagnetic radiation source can be a low-energy, green-light emitter, such as a KTP laser capable of emitting light of a wavelength of 532 nanometers. For other activating agents, the wavelength used can range from about 350 nanometers to about 800 nanometers, preferably between about 400 nanometers to about 700 nanometers.

Moving on to process block 16, illuminating the wound with the electromagnetic radiation source activates the activating agent, inducing crosslinking of the extracellular matrix. In one non-limiting example, this includes crosslinking of collagen of the extracellular matrix. More specifically, protein crosslinking naturally occurs in the body due to enzyme-catalyzed or spontaneous reactions. Disulfide bond formation is one of the most common types of crosslinking but isopeptide bond formation is also common. However, proteins may also be crosslinked artificially, such as through activating agents or chemical crosslinking agents. Here, when an activating agent is distributed adjacent to collagen, the activating agent may bind to the collagen in a noncovalent manner. The illumination then activates the activating agent to induce collagen crosslinking through covalent bonding. More specifically, photo-activation of the activating agent is a process by which the electromagnetic irradiation is absorbed by the agent, thus raising the compound to an electronically excited state. The excited compound then uses the additional energy to fuel chemical reactions that are responsible for bond formation such as protein crosslinking within the tissue. Furthermore, while other structural proteins like elastin may not have the same physical interaction with the activating agent as collagen, these other proteins may still experience the same crosslinking response to the illumination as collagen.

At process block 18, the effect of crosslinking is to produce wound healing that better matches normal tissue, for example, in color, texture, thickness, and/or function (that is, compared to untreated wounds). In other words, the crosslinking caused by the activating agent causes thicker, more organized collagen fibers, increased ingrowth and development of dermal cells, increased vascularity, appearance of skin appendages (e.g., hair follicles, sebaceous glands, sweat glands, etc.) earlier and to a greater degree, decreased contracture, and less scarring compared to untreated healed wounds. Furthermore, one mechanism by which contracture may be reduced through crosslinking of the matrix is that such crosslinking reduces the ability of fibroblasts and myofibroblasts to migrate into the wound. In addition, crosslinking of the matrix provides mechanical resistance to contractile forces exerted by myofibroblasts on the tissue (which cause scar contracture).

In some aspects, the above method 10 may be repeated more than once throughout the wound healing process. For example, the method 10 may be repeated daily, weekly, or at another suitable continuous or variable interval. Additionally, the method 10 may be repeated for a set duration, until the wound is closed, or until the wound is fully healed.

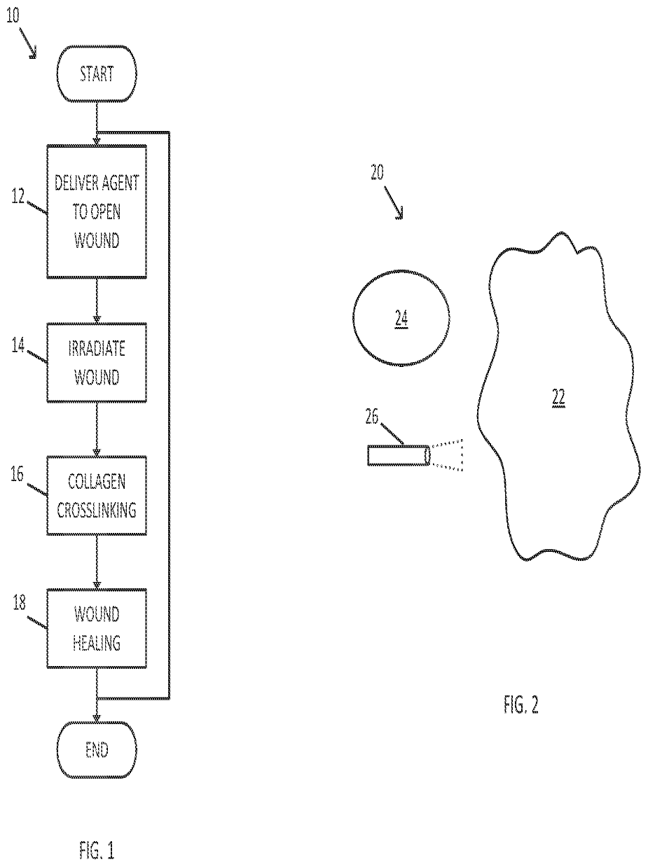

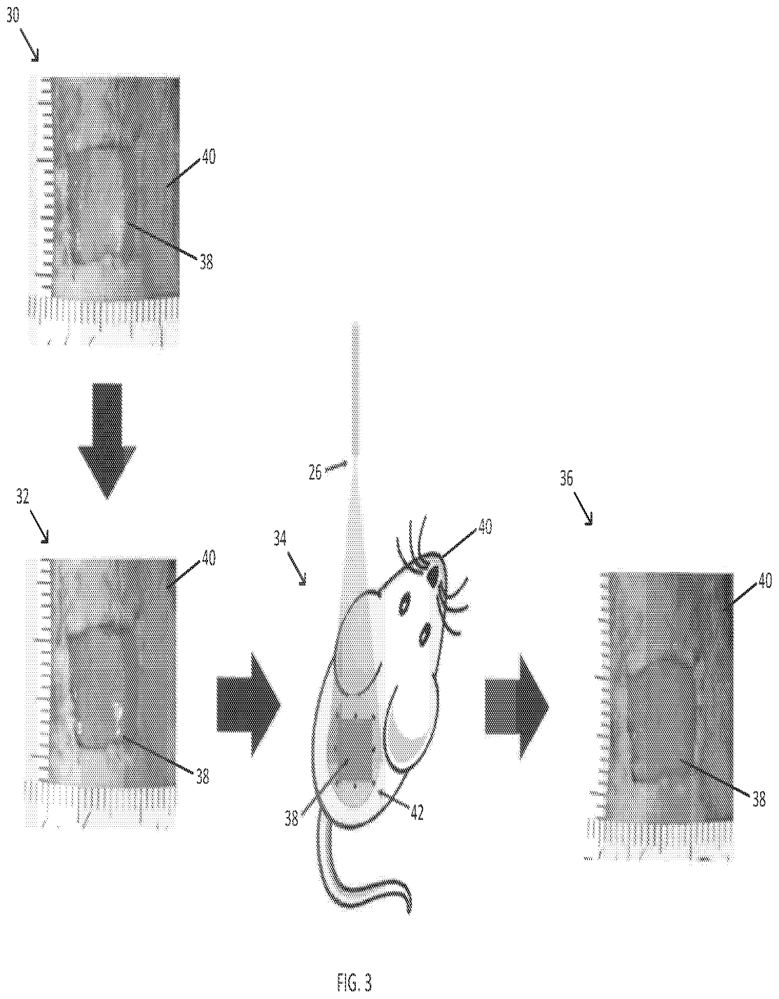

FIG. 2 is a schematic view of an example system 20 according to one aspect of the disclosure. The system 20 can be used to treat a wound 22 according to the method 10 of FIG. 1, that is, to facilitate optimal wound healing. The system 20 generally includes a delivery mechanism 24 and an electromagnetic radiation source 26. The delivery mechanism 24 may be one or more applicators, such as sponges, brushes, cotton tip applicators, needles, or other suitable applicators. Additionally, as described above, an applicator can be a material, such as a pre-treated bandage, containing the activating agent. The electromagnetic radiation source 26 may include a light-emitting system, such as a light emitting diode (LED), laser, or other suitable radiation source, such as any of the examples described above.

By way of example, the above-described system 20 and method 10 were studied in comparison to untreated control wounds. The study was carried out according to the steps illustrated in FIG. 3. Generally, FIG. 3 illustrates a wound creation step 30, an activating agent delivery step 32, an illumination step 34, and a post-treatment step 36. More specifically, at step 30, full-thickness, excisional 1 cm by 1 cm wounds 38 were created on the dorsa of C57BL/6 mice 40 in both a control group of 16 mice and a test group of 16 mice. Additionally, dots 42 were tattooed on the skin around the wound perimeter in both the control and test groups (as shown in step 34) in order to monitor contracture over the course of wound healing. Following step 30, control group wounds were left alone to heal. With respect to the test group, at step 32, a Rose Bengal solution was painted onto wound beds 38 (e.g., as discussed above with respect to process block 12 of FIG. 1). At step 34, the wounds 38 were illuminated by an electromagnetic radiation source 26, in particular, a KTP laser having a 532-nanometer wavelength, with an energy output of 60 Joules per centimeter squared (e.g., as discussed above with respect to process block 14 of FIG. 1). Steps 32 and 34 were conducted immediately after wound creation at step 30. Step 36 illustrates the immediate result of wound treatment, that is, the photobleaching of Rose Bengal dye.

To compare the test and control groups, the area within each tattooed wound perimeter was serially measured over 6 weeks and percent contracture was calculated. Additionally, at 7, 14, 21, and 42 days, mice were euthanized and tissue was harvested for histology.

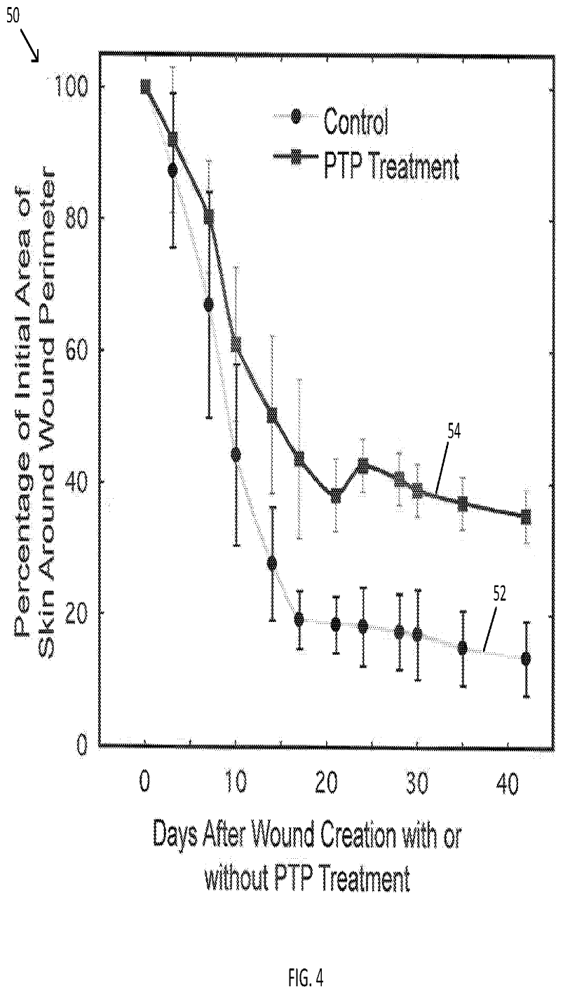

At the end of the study, all wounds were fully healed. However, the control wounds exhibited almost 20% more contracture by day 7 (67.1±17.1% in the test group versus 80.3±8.5% in the control group; p=0.014, n=16 mice per group). In particular, FIG. 4 illustrates a chart 50 of percentage of initial area of skin around wound perimeter (e.g., as defined by the tattooed dots, starting at 100% at day zero) as a function of days after wound creation, for both the control group (line 52) and the test group (line 54). As shown in FIG. 4, the degree of contracture plateaued for both groups around week three. By day 42, in the control group 52, wounds had contracted to 13.6±5.6%, whereas in the test group 54, wounds had only contracted to 35.2±2.9% (a 1.59-fold difference, p=0.003). Accordingly, the present systems and methods described herein reduce wound contracture during healing of secondary intention wounds, as compared to natural healing.

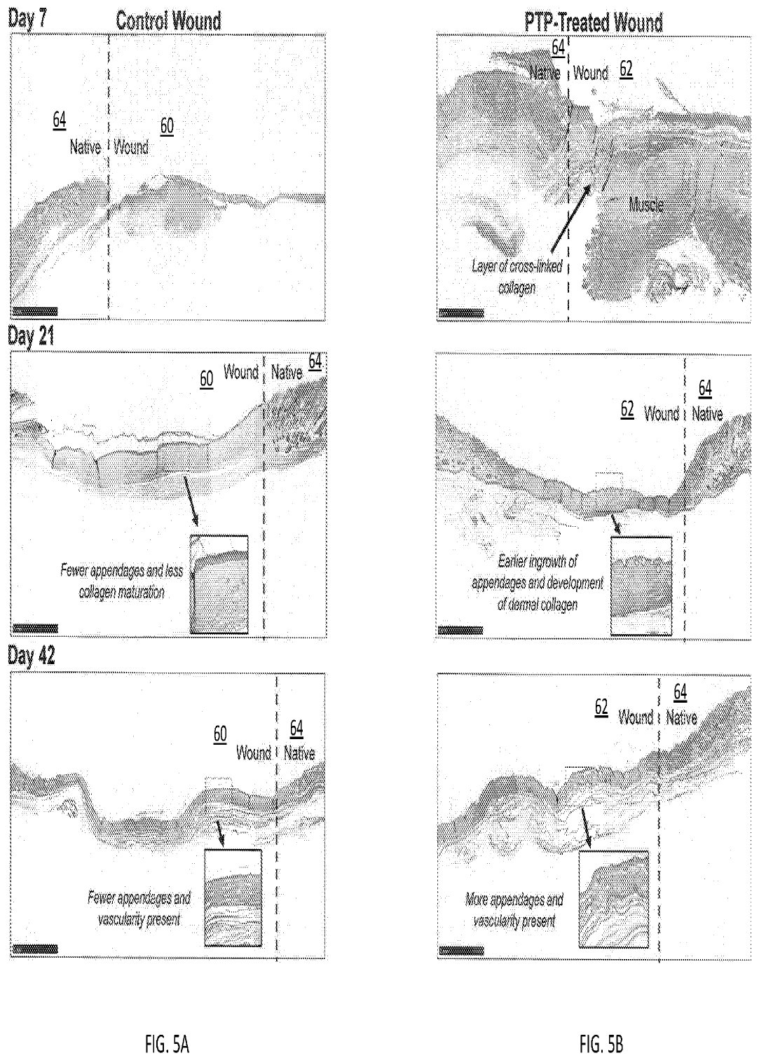

Additionally, FIGS. 5A and 5B illustrate tissue histology of control group wound tissue 60 and test group wound tissue 62, respectively, adjacent normal native tissue 64 at days 7, 21, and 42 post wound creation. Upon histological review, as shown in FIG. 5, treatment in the test group tissue 62 caused increased ingrowth and development of dermal cells, increased vascularity, and appearance of skin appendages earlier and to a greater degree, compared to the control group tissue 60. Accordingly, the present systems and methods described herein improve healing of secondary intention wounds by manipulating the wound healing response to better match structure and function of normal tissue, as compared to a naturally healed wound.

In light of the above, the systems and methods described herein for photochemical treatment of epithelial wounds inhibits wound contracture, facilitates earlier wound maturation, and results in more normal tissue production (e.g., earlier appearance of skin appendages and dermal collagen and to a greater degree). As a result, the present systems and methods facilitate more optimal wound healing that results in tissue better matching surrounding normal tissue. Furthermore, in contrast to previous applications, which involve crosslinking an internal, closed tissue surface to strengthen the tissue, the present methods are applied to wounds, not for changing a mechanical strength of the tissue, but rather to manipulate the wound healing response. For example, rather than strengthening a closed tissue surface, the present methods may be used on a wound to create an autologous scaffold to promote wound healing.

Accordingly, in some aspects, the present systems and/or methods can be applied to full thickness or partial thickness excisional wounds to improve wound healing (including reducing scarring and preventing contracture). In other aspects, the present systems and/or methods can be applied to any raw, wounded, or injured skin tissue to improve wound healing. Furthermore, in some aspects, the treated wound can be supplemented with cells or growth factors to accelerate wound healing. In other words, such cells or growth factors may be applied to the wound before or after treatment. For example, cells such as epithelial cells, stromal cells, adipocytes, adipose derived stem cells, smooth muscle cells, melanocytes, stem cells, endothelial progenitor cells, and/or blood and immune cells may be applied to the wound. Treated wounds can be treated with stromal vascular fraction, platelet rich plasma, fibrin, platelet-derived growth factor, (TFG)-β, fibroblast growth factor, or epidermal growth factor.

Furthermore, in some aspects, the present systems and/or methods may be applied to an open wounds containing (e.g., covered by) a tissue graft to improve and/or accelerate wound healing. For example, contractile response is a common complication associated with grafted tissue, such as split-thickness grafts. Thus, the present methods, which inhibit contractile response, may result in a healed graft that better resembles surrounding normal tissue. Accordingly, in some aspects, the wound bed could be covered with a protective graft(s) such as a split thickness skin graft, a full thickness skin graft, an epidermal graft, a dermal graft, a basement membrane graft, a fascia graft, an adipose graft, an acellular dermal graft, a xenograft, a subintestinal submucosa graft, a collagen graft, a silicone graft, an amniotic membrane graft, an alginate graft, a silk graft, and/or a hydrogel graft. These grafts may be applied as a continuous sheet, a collection of cores, or a collection of morselized material. The graft(s) may be placed on top of a treated wound bed or the grafts themselves could be treated. In another aspect, both the wound bed and the graft may be treated. Alternatively, the present systems and methods may be used for wound healing as a replacement for tissue grafting.

In some applications, the present systems or methods may be applied to internal epithelial tissue wounds to prevent contracture and/or promote healing. Such wounds may include ulcers or other wounds of the digestive track (or other internal epithelial tissue). For example, endoscopic mucosal resections, endoscopic submucosal dissections, or other procedures of lumen of the digestive track result in an open wound. Natural healing of such wounds has a risk of scarring and contracture, which may result in narrowing the lumen (i.e., stricture). When such stricture occurs in the esophagus, the subject may experience difficulty swallowing and require additional treatment to resolve this issue. On the other hand, the present methods may be applied after such procedures to reduce the risk of wound contracture during healing and, thus, reduce the risk of esophageal stricture. Accordingly, the present systems and methods, by reducing the risk of contracture, may further reduce the risk of stricture when applied to wounds of internal lumen. Additionally, in some aspects, photochemical crosslinking for the purpose of tissue strengthening may also be applied during such procedures to help reduce the risk of tissue perforation.

The present invention has been described in terms of one or more preferred embodiments, and it should be appreciated that many equivalents, alternatives, variations, and modifications, aside from those expressly stated, are possible and within the scope of the invention. Furthermore, the term “about” as used herein means a range of plus or minus 20% with respect to the specified value, more preferably plus or minus 10%, even more preferably plus or minus 5%, most preferably plus or minus 2%. In the alternative, as known in the art, the term “about” indicates a deviation, from the specified value, that is equal to half of a minimum increment of a measure available during the process of measurement of such value with a given measurement tool.

1. A method for improving healing of an epithelial tissue wound, the method comprising the steps of:
delivering an activating agent to the wound;
irradiating the wound with an electromagnetic radiation source; and
activating the activating agent, in response to the irradiation, to cause extracellular matrix crosslinking throughout the wound.

2. The method of claim 1, wherein the step of delivering includes topically applying the activating agent to a surface of the wound.

3. The method of claim 2, wherein the step of applying includes using an applicator to apply the activating agent to the wound, and wherein the applicator is one of a sponge, a brush, a cotton tip applicator, a needle, and a bandage.

4. The method of claim 1, wherein the step of irradiating is performed for a duration between about one minute to about thirty minutes.

5. The method of claim 1, wherein the step of irradiating is performed for a duration of less than about five minutes.

6. The method of claim 1, wherein the step of irradiating is performed using the electromagnetic radiation source having a wavelength between about 350 nanometers to about 800 nanometers.

7. The method of claim 1, wherein the step of irradiating is performed using the electromagnetic radiation source having a wavelength between about 400 nanometers to about 700 nanometers.

8. The method of claim 1, wherein the activating agent is one of a xanthene, a flavin, a thiazine, a porphyrin, an expanded porphyrin, a chlorophyll, a phenothiazine, a cyanine, a mono azo dye, an azine mono azo dye, a rhodamine dye, a benzophenoxazine dye, an oxazine, an anthroquinone dye, Rose Bengal, erythrosine, riboflavin, methylene blue, Toluidine Blue, Methyl Red, Janus Green B, Rhodamine B base, Nile Blue A, Nile Red, Celestine Blue, Remazol Brilliant Blue R, riboflavin-5-phosphate, and N-hydroxypyridine-2-(I H)-thione.

9. The method of claim 1, wherein the step of irradiating includes using one of a laser, a lamp, a light-emitting diode, and a light-emitting diode array.

10. The method of claim 1, wherein the wound contains a tissue graft.

11. The method of claim 1, wherein the wound is a full thickness skin wound.

12. The method of claim 1, wherein the wound is a partial thickness skin wound.

13. The method of claim 1, wherein the step of irradiating is performed at an irradiance of less than about one watt per centimeter squared (W/cm²).

14. The method of claim 1 and further comprising repeating the steps of delivering, irradiating, and activating periodically until the wound is closed.

15. The method of claim 1 and further comprising repeating the steps of delivering, irradiating, and activating periodically until the wound is fully healed.

16. The method of claim 1 and further comprising applying a tissue graft to the wound after the steps of delivering, irradiating, and activating.

17. The method of claim 16 and further comprising:
delivering the activating agent to the tissue graft; and
irradiating the tissue graft with the electromagnetic radiation source.

18. The method of claim 1 and further comprising applying one of cells and growth factors to the wound after the steps of delivering, irradiating, and activating.

19. The method of claim 18, wherein the cells include one of epithelial cells, stromal cells, adipocytes, adipose derived stem cells, smooth muscle cells, melanocytes, stem cells, endothelial progenitor cells, blood cells and immune cells.

20. The method of claim 1 and further comprising treating the wound with one of stromal vascular fraction, platelet rich plasma, fibrin, platelet-derived growth factor, (TFG)-β, fibroblast growth factor, and epidermal growth factor after the steps of delivering, irradiating, and activating.
